(19) United States
(12) Patent Application Publication
(10) Pub. No.: US 2017/0281530 A1
(43) Pub. Date: Oct. 5, 2017

(54) SYSTEMS AND METHODS FOR PRODUCING HOMOGENOUS PHARMACEUTICAL COMPOSITIONS

(71) Applicant: THE COMPOUNDERS DEPOT, INC., Lucedale, MS (US)

(72) Inventors: Michael Bennett, Hattiesburg, MS (US); Darian Chandler, Pace, FL (US); John Neraas, Butte, MT (US); Alton Samuel Kelley, II, Lucedale, MS (US)

(73) Assignee: The Compounders Depot, Inc., Lucedale, MS (US)

(21) Appl. No.: 15/479,674

(22) Filed: Apr. 5, 2017

Related U.S. Application Data
(60) Provisional application No. 62/318,645, filed on Apr. 5, 2016.

(51) Int. Cl. A61K 9/00; B01F 3/08; A61K 47/18; A61K 47/10; A61K 47/06

(52) U.S. Cl. CPC A61K 9/009; A61K 47/10; A61K 47/06; A61K 47/18; B01F 3/0803; B01F 3/0811

(57) ABSTRACT

Embodiments of the present disclosure generally relate to systems and methods for mixing pharmaceutical compositions, agents and/or ingredients together. In one embodiment, a method can include a shell and a flexible pouch disposed within the shell. The flexible pouch can include at least one active pharmaceutical ingredient and at least one delivery agent. Further, the flexible pouch and the shell can be configured to receive a dispensing member for dispensing a predetermined amount of a pharmaceutical composition. Methods can also include subjecting the container to high intensity vibrations for a predetermined mixing time to produce the pharmaceutical composition.

### PRIORITY

This U.S. non-provisional application claims the benefit under 35 USC §119(e) to U.S. provisional application Ser. No. 62/318,645 filed on Apr. 5, 2016, which is incorporated herein by reference in its entirety for all purposes.

### TECHNICAL FIELD

Embodiments of the present disclosure relate to systems and methods for combining active pharmaceutical ingredients and delivery agents. More specifically, the embodiments disclosed herein related to systems and methods for combining active pharmaceutical ingredients and delivery agents using high intensity vibrations in order to produce nearly homogenous to homogenous pharmaceutical compositions.

### BACKGROUND

Many pharmaceutical compositions, from orally administered drugs to topically applied creams, contain ingredients beyond the active pharmaceutical ingredient (“API”). For example, in addition to the API, the pharmaceutical products can include delivery agents, such as fillers, stabilizers, disintegrants, absorption control agents, taste maskers, and/or viscosity control agents, among many others. To produce the pharmaceutical compositions, mixing and/or blending the API with the one or more delivery agents is commonly done using a large container. After the API is mixed and/or blended with the one or more delivery agents, the pharmaceutical composition is then distributed into small containers that are sold to end consumers.

### SUMMARY

Embodiments of the disclosure relate to systems and methods for producing homogenous compositions, for example pharmaceutical compositions or formulations.

In one embodiment, a method can include receiving a container that includes a shell and a flexible pouch disposed within the shell, where the flexible pouch has at least one active pharmaceutical ingredient or agent and at least one delivery agent, and where the flexible pouch and the shell can be configured to receive a dispensing member for dispensing a metered (predetermined or aliquoted) amount of a pharmaceutical composition; and subjecting the container to high intensity vibrations for a mixing time to produce the pharmaceutical composition.

In another embodiment, a pharmaceutical composition with a high geometric dilution, prepared by a process can include: placing at least one active pharmaceutical ingredient into a flexible container, where the flexible container can be disposed within a shell and where the flexible container and the shell form an opening configured to receive a dispensing member for dispensing a metered (aliquoted or predetermined) amount of a pharmaceutical composition; placing at least one delivery agent into the flexible container; and subjecting the flexible container and the shell to high intensity vibrations for a predetermined mixing time, where the at least one active pharmaceutical ingredient and the at least one delivery agent form the pharmaceutical composition or formulation.

In another example, a method comprises: receiving a container comprising a flexible pouch disposed within a rigid container, wherein the flexible pouch comprises an opening aligned with an opening of the rigid container and wherein the flexible pouch comprises at least one active pharmaceutical ingredient and at least one delivery agent; and subjecting the container to high intensity vibrations for a mixing time to produce a pharmaceutical composition or pharmaceutical formulation.

While multiple embodiments are disclosed, still other embodiments of the disclosed subject matter will become apparent to those skilled in the art from the following detailed description, which shows and describes illustrative embodiments of the disclosure. Accordingly, the drawings and detailed description are to be regarded as illustrative in nature and not restrictive.

### BRIEF DESCRIPTION OF THE DRAWINGS

FIG. 1 depicts an illustrative system for producing a pharmaceutical composition, in accordance with some embodiments of the disclosure.

FIGS. 2A-2B depict an illustrative container that can be used in the system depicted in FIG. 1, in accordance with some embodiments of the disclosure.

FIG. 3 is a flow diagram of an illustrative method for producing a pharmaceutical composition, in accordance with some embodiments of the disclosure.

FIG. 4 is a flow diagram of another illustrative method for producing a pharmaceutical composition, in accordance with some embodiments of the disclosure.

While the disclosed subject matter is amenable to various modifications and alternative forms, specific embodiments have been illustrated by way of example in the drawings and are described in detail below. The intention, however, is not to limit the disclosure to the particular embodiments described. On the contrary, the disclosure is intended to cover all modifications, equivalents, and alternatives falling within the scope of the disclosure as defined by the appended claims.

As the terms are used herein with respect to ranges of measurements (such as those disclosed immediately above), “about” and “approximately” can be used, interchangeably, to refer to a measurement that includes the stated measurement and that also includes any measurements that are reasonably close to the stated measurement, but that can differ by a reasonably small amount such as will be understood, and readily ascertained, by individuals having ordinary skill in the relevant arts to be attributable to measurement error, differences in measurement and/or manufacturing equipment calibration, human error in reading and/or setting measurements, adjustments made to optimize performance and/or structural parameters in view of differences in measurements associated with other components, particular implementation scenarios, imprecise adjustment and/or manipulation of objects by a person or machine, and/or the like.

Although the term “block” can be used herein to connote different elements illustratively employed, the term should not be interpreted as implying any requirement of, or particular order among or between, various steps disclosed herein unless and except when explicitly referring to the order of individual steps.

### DETAILED DESCRIPTION

In certain embodiments, methods to produce pharmaceutical compositions or formulations, mixing and/or blending the API with the one or more delivery agents is typically performed using a large container. After the API is mixed and/or blended with the one or more delivery agents, the pharmaceutical composition is then distributed into small containers that are sold to end consumers. Frequently, these methods result in incomplete mixing and loss of the API and the mixed pharmaceutical composition in the process of transfer of to the small containers. Mixing these APIs to form uniform usable pharmaceutical compositions without destroying or losing some of the API is a challenging process. Adding to this complexity, agents that make up these pharmaceutical compositions can be derived from different sources that behave differently under identical conditions due to variances in certain factors such as particle size, shape, viscosity, and aggregate forming tendencies of these agents, leading to inconsistent mixing results. Therefore, providing reliable, reproducible methods for mixing APIs is sought.

In addition to the challenges described above, mixing in larger batches contains certain processing disadvantages, for example, inconsistent mixing due to the large amount of compounds being mixed. Furthermore, mixing in larger batches requires the producer to front large volumes of ingredients that could potentially expire before being used up or sold to consumers. Any process that blends a pharmaceutical product must also account for external factors such as product loss, operator exposure, and sterility control. For example, mixing large quantities of powdered ingredients that are a risk to the human operator mixing the agents increases risks of exposing the operator through inhalation, eye or skin contact. If an API is mixed in a first container then moved to a second container for packaging, a portion of the mixture can typically be lost when transferring the mixture from the first container to the second container. The incentive to limit product loss is especially acute when mixing or blending limited quantities and/or expensive APIs. Accordingly, there is a need for a mixing process that can consistently provide a uniformly mixed pharmaceutical composition, while maintaining product sterility, minimizing exposure to the operator and minimizing product loss. Embodiments described herein can provide solutions to these problems and satisfy the corresponding needs.

Throughout this disclosure, compositions produced using one or more of the embodiments described herein are described as being “homogenous.” Various methods can be used to determine the homogeneity of an API in a composition. For example, a small amount of coloring can be added to the composition to determine whether the coloring is evenly distributed. As another example, a lab can perform high-performance liquid chromatography (HPLC) or other analysis procedure on the composition in order to access homogeneity. In accordance with these embodiments, using HPLC, a lab can test the concentration of the API in different areas of the composition and then calculate the relative standard deviation (RSD) of the concentration of the API throughout the composition. While the term “homogenous” is used herein, the compositions may be near homogenous. For example, in some embodiments, the term “homogenous” as used herein may indicate a composition having an RSD less than about 6% or less than about 4%. In embodiments, some of the compositions produced using the systems and methods described herein have been tested and demonstrate a RSD of less than 0.5%.

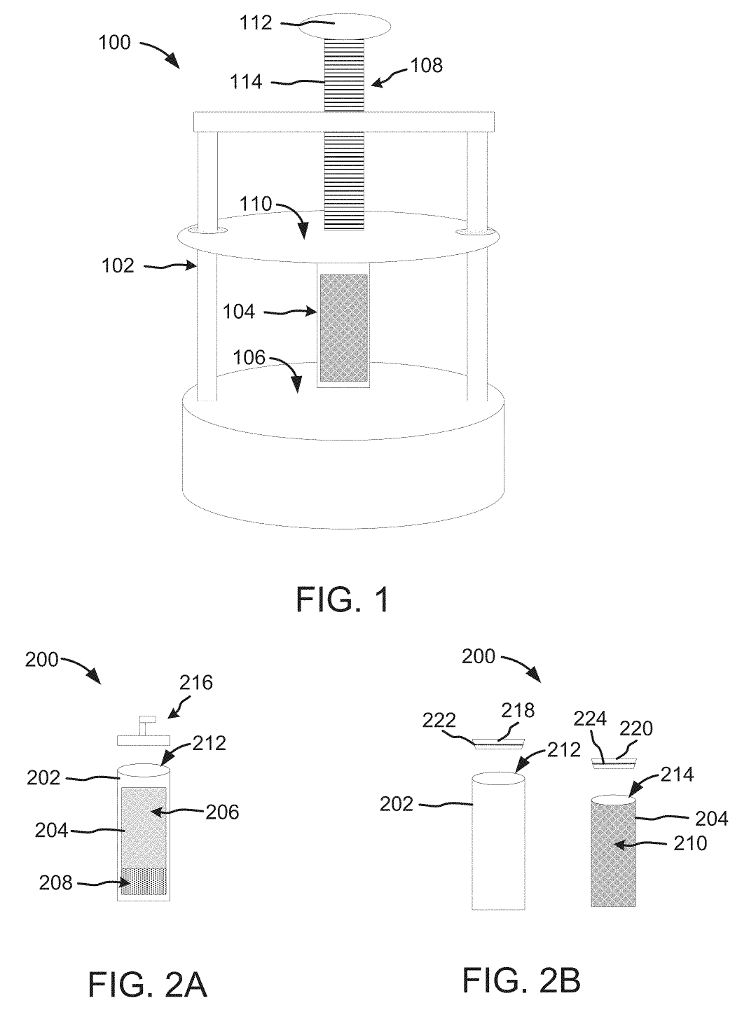

FIG. 1 depicts an illustrative system 100 for producing a pharmaceutical composition, in accordance with some embodiments of the disclosure. The system 100 includes a mixing device 102 and a container 104. In certain embodiments, the container 102 can include a first container and a second container (also depicted in FIGS. 2A-2B). The second container is insertable within the first container (also depicted in FIG. 2A). Moreover, the second container is capable of sealing an active pharmaceutical ingredient (API) and a delivery agent therein.

Once a container 104 has been filled with an API and a delivery agent, container 104 can be placed on a base 106 of the mixing device 102 and secured to the mixing device 102 using a retaining mechanism 108. In the embodiment illustrated, the retaining mechanism 108 includes a platform 110 and an actuating mechanism 112. The actuating mechanism 112 is coupled to the platform 110, via a coupler 114 (e.g., a screw). Actuating mechanism 112 is also coupled to a frame 116 that projects from the base 106. When the actuating mechanism 112 is actuated, coupler 114 provides a force on the frame 116. The force on frame 116 is translated to platform 110 via a coupler 114. The force translated to platform 110 results in movement of platform 110 in either an up or down direction relative to the base 106, depending on which direction actuating mechanism 112 is actuated. For example, actuating the actuating mechanism 112 in a clockwise direction can result in movement of platform 110 towards the base 106 while actuating actuating mechanism 112 in a counter-clockwise direction can result in movement of platform 110 away from the base 106.

Mixing device 102 depicted in FIG. 1 is only an example and is not meant to be limiting. Any other mixing device and/or variation of mixing device 102 as depicted in FIG. 1 can be used, as long as the mixing device 102 and/or the variation of the mixing device 102 is capable of imparting appropriate mechanical energy to container 104, as described below.

After container 104 is retained by mixing device 102, mixing device 102 can be turned on. Once mixing device 102 is turned, the mixing device 102 is configured to impart mechanical energy to container 104 and container's 104 contents. In some embodiments, mechanical energy can originate from base 106 and translate from base 106 to container 104. In accordance with these embodiments some examples of mechanical energy that can be imparted by mixing device 102 can include, but is not limited to, rotational energy (e.g. spinning the container), translational energy (e.g. tumbling the container), or vibrational energy (e.g. shaking or vibrating the container). In certain exemplary embodiments, the mixing device can impart vibrational energy to container 104. In other embodiments, vibrational energy can be acoustical vibrational energy.

In one embodiment, acoustic mixing device 102 can use non-contact mixing that relies on applying a low-frequency acoustic field to facilitate mixing within a container 104. In accordance with this embodiment, acoustic mixing can work based on creating micro-mixing zones throughout the entire container 104. This technique differs from more traditional techniques, such as moving a blade through the container 104 or using baffles, where the mixing zone is localized in discrete locations such as the leading edges of the blades or baffles. Acoustic mixing can create faster, more efficient and uniform mixing throughout the entire container 104. In other embodiments, acoustic mixing can be applied to a single phase or multiphase system, for example a liquid-liquid, liquid-solid, gas-liquid, or solid-solid system.

In some embodiments, acoustic mixing device 102 is designed to operate at mechanical resonance. When operating at mechanical resonance, even small periodic driving forces, such as acoustical vibrations, can produce large amplitude vibrations that can translate, in this case, to more efficient mixing. In certain embodiment, acoustic mixing device 102 imparts acoustic vibrations to the container 104 and its contents at resonant frequency. As provided herein, “resonant frequency” can refer to the natural frequency of vibration of the vibrating objects. In certain embodiments, the resonant frequency is in the range at which the maximum mechanical energy that can be imparted from the driving force is transferred to the moving mass, here the container 104 and its contents (e.g., the API 206 and the delivery agent 208 depicted in FIGS. 2A-2B) in order to create a more favorable and efficient mixing of the components of container 104. Using this operating condition reduces the energy loss and results in a more complete energy transfer to the container 104. This operation can be further optimized by matching the mechanical operating conditions of the mixing device 102 to the natural frequency of the container 104 and the properties and characteristics of the materials to be mixed.

In some embodiments, resonant frequency can be governed at least in part by the total mass of the container 104 and its contents. At resonance, inertial and stored forces of the total mass are canceled out and the total input force contributed by the acoustic mixing device 102 is imparted to the container 104 which is then translated to mixing force. The energy transfer from the container 104 to its contents at resonant frequency is then subject to the physical properties of the material in the container 104, for example, its viscosity and how well it adheres to the interior surface of the container 104. Because a liquid is able to take on the interior shape of the container 104, it has a greater total contact area with the container 104, and thus mixing energy is more efficiently transferred to a liquid in the container 104 than a solid.

In other embodiments, when at low-frequencies and high amplitude acoustic oscillations, a liquid contained in the container 104 can undergo second order bulk motion. In accordance with these embodiments, this second order bulk motion drives the liquid, which then causes acoustic streaming in the liquid. Acoustic streaming in the liquid produces a multitude of micro-mixing cells throughout the liquid. At frequencies of about 60 Hz, for example, a nominal mixing cell length is about 50 microns. With multiple mixing cells of such short length spread throughout the liquid, a more thorough mixing process is produced. And with the high efficiency in the transfer of mechanical energy into the mixing process, the blending rates may exceed conventional techniques, resulting in shorter mixing times required. In some embodiments disclosed herein by subjecting contents of a container 104 to low frequency, high intensity acoustic vibrations, the contents can be mixed to a high level of geometric dilution in a short period of time. This process can improve homogeneity of a target mixture saving time and money.

In certain embodiments, acoustic parameters such as frequency, amplitude, and intensity can be selected from suitable parameters for a particular container 104 and ingredients (e.g. pharmaceutical agents) being mixed. In other embodiments, container 104 and its contents are subjected to acoustic vibrations of approximately 10 to approximately 1,000 Hz, approximately 10 to 500 Hz, or approximately 15 to 100 Hz. In some embodiments, container 104 and its contents can be subjected to displacements of 0.01 to 1.0 inches, 0.02 to 0.5 inches, or 0.05 and 0.1 inches. In other embodiments, the container 104 and its contents can be subjected to acoustic mixing intensity of 10 to 100 gs, with one “g” referring to the force of acceleration imparted by the gravitational pull of the earth on a stationary body at sea level.

The length of time the mixing process (also referred to herein as a mixture time) is carried out is also dependent on the ingredients being mixed, the physical characteristics of the starting materials, and the desired final composition. For example, particle size, solubility, and/or viscosity, among others, are taken into account when selecting acoustic mixing parameters. For example, a mixture including a first API having a first particle size that is smaller than the particle size of a second API can be mixed for a shorter period of time and/or at a lower intensity than a mixture containing the second API. As another example, a mixture including a first delivery agent having a less viscous and/or higher solubility than a second delivery agent can be mixed for a shorter period of time and/or at a lower intensity than a mixture containing the second delivery agent. In one embodiment, an acoustic mixing device that can be suitable for mixing agents disclosed herein is a LabRAM® ResonantAcoustic® Mixer, available from Resodyn Acoustic Mixers, Inc. of Butte, Mont.

In some embodiments, a healthcare worker or pharmacist can expose the contents of the container 104 to a slight vacuum, and then subject the mixture to of about 10 to 20 gs for one minute. Using this method will remove trapped air that has been incorporated into the mixture.

A pharmacist can conduct the mixing of the mixture after receiving a prescription, in accordance with current industry procedures. In some embodiments, the mixing can be carried out by a licensed provider or FDA approved manufacturer closer to the point of distribution, rather than a pharmacist. Optionally, a pharmaceutical provider can supply a premade paste or mixture that contains the API in an appropriate delivery agent. The paste or mixture can be delivered in a multiuse and metered apparatus such as a single use package or syringe. The licensed provider or FDA approved manufacturer can perform the mixing before providing the mixed paste to the end user or healthcare provider. In some embodiments, a licensed provider or FDA approved manufacturer can source containers 104 prefilled with suitable delivery fluids in an appropriate volume. When an end user requires a particular cream in a particular concentration, the licensed provider or FDA approved manufacturer can add a suitable amount of an API to the prefilled container 104 and perform the mixing of the container 104 to create a pharmaceutical composition for the end user, for example, a patient in need of such a composition.

In some embodiments, using an acoustic mixing device 102 allows a healthcare worker, pharmacist, patient or other to avoid the product losses suffered with alternative mixing means such as an electronic mortar and pestle or an ointment mill. The systems and methods disclosed herein can minimize the chance of cross contamination with other APIs by eliminating the use of equipment that has been used for mixing other products. Mixing each prescription within designated packaging that will be used to dispense the prescription provides a healthcare worker with individual batches in a specific amount for a subject in need.

In accordance with these embodiments, the API and delivery agent can be mixed by the mixing device 102 until the API and delivery agent form a geometrically diluted composition such as a cream, paste or gel. As used herein, the term “geometrically diluted cream” can include a cream that is a compounded mixture that contains one or more APIs and has the physical, tactile, and visual characteristics of a cream with liquid-like homogeneity.

FIGS. 2A-2B depict an illustrative container 200 that can be used in the system 100 depicted in FIG. 1, in accordance with some embodiments of the disclosure. In certain embodiments, the container 200 includes a first container 202 and a second container 204. As stated above, the second container 204 is insertable within the first container 202 (as illustrated in FIG. 2A). Moreover, the second container 204 is capable of receiving an API 206 and a delivery agent 208 therein. In some embodiments, the API 206 can be in a powdered, pelleted, crushed, solid, liquid, gel-like, cream, emulsion, or suspension form, or any combination or variation thereof. An API 206 that will be mixed can be weighed, recorded, and placed within the second container 204. The amount or volume of API 206 placed in the second container 204 can depend on a prescription or required concentration. For example, the API 206 amount can be a specifically prescribed amount calculated to create a cream at a specified or pre-determined concentration for a particular patient or subject. In some embodiments, the API 206 is in a pelletized, crushed, liquid, or powdered form prior to mixing.

The delivery agent 208 can be an ointment, a liquid carrying agent, a levigating agent, or any combination or variation thereof. Similar to the API 206, the delivery agent 208 can be weighed, recorded, and placed inside the second container 204. As used herein, the term “delivery agent” can refer to any liquid, gel, ointment, or cream that can be mixed with an API 206 to form a target or desired pharmaceutical composition 210 that will maintain the API 206 in a physically and chemically stable condition at least until the pharmaceutical composition 210 is applied by an end user to a patient or subject. In some embodiments, the delivery agent 208 is selected from liquids that will not chemically react with the API 206. The delivery agent 208 can optionally be selected from any liquid that will adhere to a end user long enough for the API 206 to be therapeutically effective. For example, the delivery agent 208 can be selected for its ability to withstand sweat or sunlight. Some suitable delivery agents 208 can include but are not limited to glycerin, propylene glycol, mineral oil, trolamine, or the emulsifying fluid sold under the trade name Emulisiflix® or any other delivery liquid known in the art.

In other embodiments, the pharmaceutical composition 210 can be any one of an emulsion, a suspension, or a solution. For example, in some embodiments, the pharmaceutical composition can 210 can be one of a gel, a paste, a dense liquid or a cream. It is not critical that the API 206 dissolve into the delivery agent 208 so long as the delivery agent 208 can retain and deliver the API 206 to the end user in a manner that does not appreciably deplete its efficacy.

As described above, the container 200 is subjected to mechanical energy for a mixing time to mix the API 206 with the delivery agent 208 to produce a pharmaceutical composition 210.

The first container 202 can be rigid, semi-rigid, semi-flexible and/or resilient walls, or be made of a flexible and non-resilient material. For example, the first container 202 can be a solid structure with a top, bottom, and walls extending between the top and bottom. However, this is only an example and not meant to be limiting.

The second container 204 can be made of a flexible material such as a plastic or polymer material formed into a seamless pouch or bag. In some embodiments, the first container 202 and/or the second container 204 is made of a resistant material such as tear resistant and/or resistant to microbial growth etc. However, this is only an example and not meant to be limiting.

In some embodiments, the first container 202 and the second container 204 include respective openings 212, 214. The opening 212 of the first container 202 can be of a size and shape that allows the second container 204 to be insertable into the first container 202. The opening 214 of the second container 202 can be of a size and shape that allows an API to be insertable into the second container 204.

In some embodiments, the openings 212, 214 are aligned so that a dispensing member 216 can be connected to the top of the container 200. In accordance with these embodiments, an example of a dispensing member 216 is a cap or spout that can be closed or opened, allowing a user to open the cap or spout, dispense the container's 200 contents, and reclose the cap or spout. If the container 200 is formed from a flexible or supple or moldable material, the user can control the amount dispensed by putting pressure on or squeezing the container 200. Optionally, a dispensing member 216 can include a pump, such as a hand driven dosing pump that uses a mechanical device such as a piston or diaphragm to draw and dispense the contents of the container 200, often in discrete metered amounts. In certain embodiments, using a mechanical dispensing member 216 such as a dosing pump allows a user to more accurately control the amount dispensed. Using a container 200 that includes a dispensing member 216 allows a healthcare worker to provide a mixed product to the end user without needing to transfer the mixed product from the container 200 to a separate dispensing container. An example of a container that can be of use for certain embodiments can be a Topi-Pump® bottle, for example, available from TCD, Inc. of Lucedale, Miss. Using a dosing pump as the dispensing member 216 and a flexible pouch as a second container 204, greater than 93% evacuation of the pharmaceutical composition 210 has been realized.

In some embodiments, before the container 200 is mixed using the mixing device 102, the openings 212, 214 may be sealed using one or more sealing members 218, 220. In embodiments, each opening 212, 214 may have a separate sealing member 218, 220. Alternatively, a single sealing member 218, 220 may seal both the openings 212, 214. In embodiments, one or both of the sealing members 218, 220 may have rings 222, 224 encircling the sealing members 212, 214. The rings 222, 224 may increase the ability of the sealing members 218, 220 to seal the opening 212, 214.

After the API 206 and the delivery agent 208 are mixed into the pharmaceutical composition 210, the sealing members 218, 220 can be removed and the dispensing member 216 can be connected to the container 200. In accordance with these embodiments, the sealing members 218, 220 can be made of a resilient material. For example, the sealing members 218, 220 can be made of silicon, rubber, cork, Teflon and/or the like.

An advantage of the container 200 depicted in FIGS. 2A-2B over conventional containers is that the container 200 is less likely to leak when the API 206 and the delivery agent 208 are being mixed together. In addition, if a different mechanism than the dispensing member 216 were used to dispense the pharmaceutical composition 210, for example, a plate mechanism that is actuated and pushes the contents of the container up from the bottom of the container, the contents of the container would likely leak when the container is being mixed by the mixing device 102 because of the seam between the sidewalls of the container and the plate mechanism. To the contrary, because the second container 204 has a single opening 214 and no seams, and because the ability to obtain a quality seal of the openings 212, 214 is better than the ability to seal a container that includes a sliding bottom plate, the API 206 and the delivery agent 208 are less likely to leak from the second container 204 when the container 200 is being mixed using the mixing device 102. Furthermore, because the pharmaceutical composition 210 can be mixed and dispensed from the container 200, there will be reduced product loss than using conventional techniques where a large batch of a pharmaceutical composition is produced using a large container and dispensed into smaller containers.

In some embodiments, the first container 202 and the second container 204 can be selected based on the type of API 206, delivery agent 208 and/or the pharmaceutical composition 210. For example, the volume of the first container 202 and the second container 204 can be based on the type of API 206, delivery agent 208 and/or the pharmaceutical composition 210. For example, if a quantity of a specific pharmaceutical composition 210 is regularly prescribed, the container 200 can have the same or similar volume as the quantity of the specific pharmaceutical composition 210 that is regularly prescribed. In some embodiments, the container 200 can also be selected based on how capable the container 200 is at retaining contents of the pharmaceutical composition 210.

In some embodiments, a healthcare worker or pharmacist can select a suitable container 200 that will also function as a dispensing container. The healthcare worker measures a dose of API 206 suitable to meet a patient's required dose or concentration. The healthcare worker selects a delivery agent 208 suitable for the particular API 206. The healthcare worker can measure a suitable amount of delivery agent 208 that will create a pharmaceutical composition 210 at the necessary concentration. After placing the measured amount of API 206 and delivery agent 208 into the container 200, the healthcare worker can seal the container 200 and place it into the mixing device (e.g., the mixing device 102 depicted in FIG. 1). After subjecting the container 200 and its contents to mixing for a suitable length of time to create an elegant and aesthetic mixture with high geometric dilution, the healthcare worker can remove the container 200 from the mixing device. The container 200 can then be labeled and distributed to the end user. If the container 200 is made up of a first container 202 and a second container 204, for example, a pouch within a solid container, the healthcare worker has an option of removing the pouch from the solid container and distributing only the pouch to the end user.

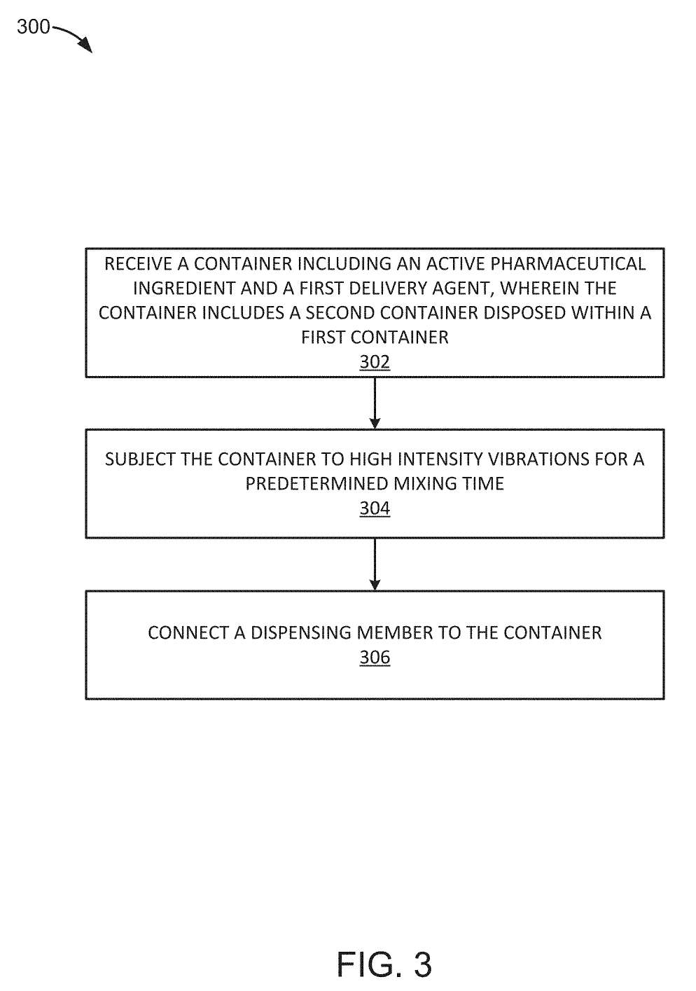

FIG. 3 is a flow diagram of an illustrative method 300 for producing a pharmaceutical composition, in accordance with embodiments of the disclosure. The method 300 includes receiving a container (block 302). In some embodiments, the container includes a second container disposed within a first container, wherein an API and a first delivery agent are disposed within the second container. In some embodiments, method 300 can include placing the API and delivery agent into the second container. After which, the method 300 can include placing the second container inside the first container and sealing the first container and/or second container using one or more sealing members. In some embodiments, the container, the first container, the second container, the API, the delivery agent and the one or more sealing members can have some or all of the same characteristics as the container 104, 200, the first container 202, the second container 204, the API 206, the delivery agent 208 and the sealing members 218, 220, respectively, described in FIGS. 1-2B above. For example, the first delivery agent can be at least one of: glycerin, ethylene glycol, propylene glycol, mineral oil, trolamine and Emulsifix®.

The method 300 also includes subjecting the container to high intensity vibrations for a first mixing time (block 304). In some embodiments, high intensity vibrations and the mixing time can be the same or similar to the high intensity vibrations and the mixing times, respectively, described in FIGS. 1-2B above. For example, the high intensity vibrations can be high intensity acoustical vibrations. As another example, the high intensity vibrations can be 10 gs to 100 gs and have a frequency of 15 Hz to 1,000 Hz. In yet other embodiments, mixing time can be 1 to 10 minutes.

Additionally or alternatively, the method 300 may include connecting a dispensing member to the container after the container is subjected to high intensity vibration (block 306). In embodiments, wherein the container includes a second container disposed within a first container, connecting a dispensing member to the container may include connecting the dispensing member to the first container, to the second container and/or to both the first and second containers. In embodiments, the dispensing member may have some or all of the same characteristics as the dispensing member 216 described above in relation to FIG. 2. In some embodiments, where the container includes a first container and a second container, the method 300 can include removing the second container from the first container and then connecting the dispensing member to the second container.

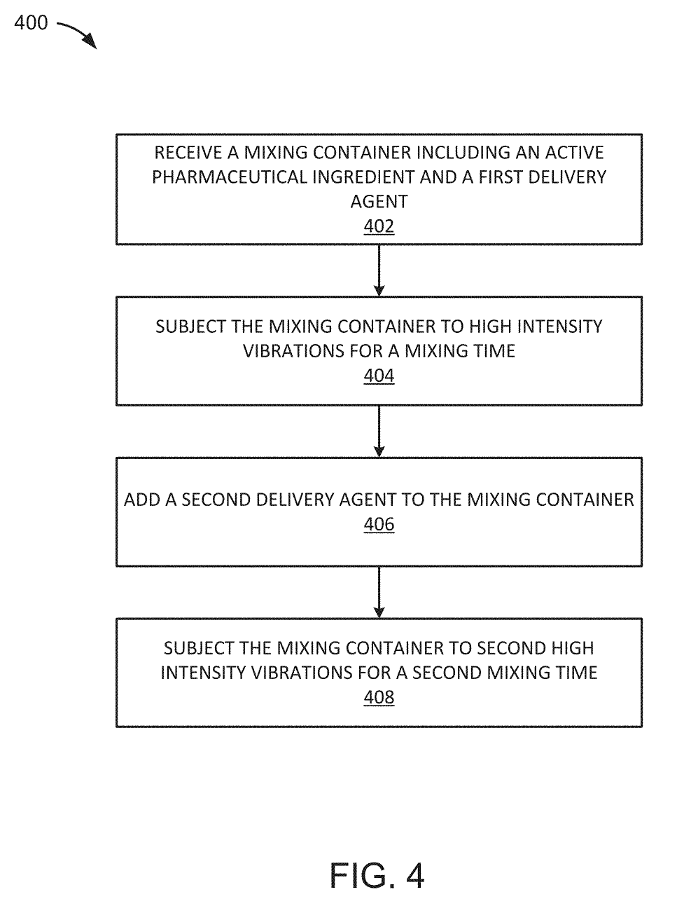

FIG. 4 is a flow diagram of another illustrative method 400 for producing a pharmaceutical composition, in accordance with some embodiments of the disclosure. Method 400 includes receiving a container (block 402). The container includes at least one API and a delivery agent. In certain embodiments, the container, the API and the delivery agent can have the same or similar characteristics the container 104, 200, the API 206 and the delivery agent 208, respectively, described in FIGS. 1-2B above. For example, the container can include a first container and a second container. As another example, the first delivery agent can be at least one of: glycerin, ethylene glycol, propylene glycol, mineral oil, trolamine and Emulsifix®.

The method 400 further includes subjecting the container to high intensity vibrations for a mixing time to produce a pharmaceutical composition (block 404). In some embodiments, the high intensity vibrations, the mixing time and the pharmaceutical composition can have the same or similar characteristics as the high intensity vibrations, the mixing time and the pharmaceutical composition 210, respectively, described above in FIGS. 1-2B. For example, the pharmaceutical composition can be one of a gel, a paste, a dense liquid or a cream. As another example, the high intensity vibrations can be high intensity acoustical vibrations. As even another example, the high intensity vibrations can be 10 gs to 100 gs and have a frequency of 15 Hz to 1,000 Hz. As even another example, the mixing time can be 1 to 10 minutes.

The method 400 also includes adding a second delivery agent to the container (block 406) and subjecting the container to second high intensity vibrations for a second mixing time to produce a second pharmaceutical composition (block 408). In some embodiments, the second delivery agent can be the same or similar to the delivery agent. Furthermore, in some embodiments, the second delivery agent can be the same or similar to the delivery agent 208 discussed above in FIGS. 1-2B above. For example, the second delivery agent can be at least one of: glycerin, ethylene glycol, propylene glycol, mineral oil, trolamine and Emulsifix®.

In some embodiments, the high intensity vibrations can be the same or similar to the second high intensity vibrations. Similarly, in some embodiments, the mixing time can be similar to the second mixing time. Furthermore, in some embodiments, the second high intensity vibrations and the second mixing time can be the same or similar to the high intensity vibrations and the mixing times, respectively, described above in FIGS. 1-2B. For example, the second high intensity vibrations can be high intensity acoustical vibrations. As another example, the second high intensity vibrations can be 10 gs to 100 gs and have a frequency of 15 Hz to 1,000 Hz. In yet other embodiments, the second mixing time can be 1 to 10 minutes.

### EXAMPLES

Embodiments of the disclosure are further defined in the following non-limiting Examples. It should be understood that these Examples, while disclosing specific embodiments, are given by way of illustration only. From the above discussion and these Examples, one skilled in the art can ascertain the essential characteristics of the embodiments of this disclosure, and without departing from the spirit and scope thereof, can make various changes and modifications of the embodiments to adapt it to various usages and conditions. Thus, various modifications of the embodiments disclosed herein will be apparent to those skilled in the art from the foregoing description. Such modifications are also intended to fall within the scope of the appended claims.

An example mixture that acoustic mixing has been shown to work effectively on is a powder-liquid mixture. Using an acoustic mixing device, a powder can be effectively and efficiently mixed with a liquid delivery fluid to create a cream with liquid-like homogeneity. The following examples were prepared by adding the ingredients individually and then vibrated in a Resodyn® acoustic mixer, in accordance with the standard operating procedure of these acoustic mixers. The compositions were determined to be successfully formed when a consistently textured, elegant and aesthetic mixture was produced.

### Example 1

In one example, a metered dose airless pouch packaging known as a Sleekline 30 was prefilled with 19.6 ml of a suitable of delivery medium (in this example Versabase® was used). The closure device (a silicone stopper) was removed and 0.4 ml of a previously prepared and provided Estradiol aliquot (100 mg/ml) is introduced into the pouch containing the delivery medium. By calculations, this mixture will result in a topical compounded medication that has an Estradiol concentration of 2 mg/ml. The closure device is restored and the stoppered Sleekline is placed securely into the Resonant acoustic mixer (RAM). The RAM unit was operated at 70G at a frequency approaching 60 Hz for 2 minutes. After which the Sleekline was removed and a 0.25 ml actuator was installed to the Sleekline bottle and pouch. The Sleekline bottle was labeled as to its contents and other information dictated by common pharmacy practices and sent to an independent laboratory to assess potency and percentage of Relative Standard Deviation (RSD). The results from the lab as it analyzed the first full actuation, the thirty-first actuation and the seventy-first actuation were the 1.88 mg/ml, 1.86 mg/ml and 1.858 mg/ml (respectively). Which results in a RSD of 0.47%, which superiorly meets the USP requirement of being less or equal to 4%.

### Example 2

In another example, a metered-dose airless pouch packaging known as a 60 ml clear Topi-pump® was prefilled with 24 ml of an appropriate delivery medium (in this example a lipodermic base). To this composition was added 120 mg of Clonidine HCl, 6 grams of Gabapentin, 6 grams of Ketoprofen, 3 grams of Lidocaine and 1.8 grams of Tramadol. The loose powders and crystals were followed by adding 6 ml of Emulsifix® (a thickening agent). The Topi-pump was sealed using a silicone stopper and placed securely in the Resonant Acoustic Mixer (RAM). The RAM unit ran at 70G at appropriately 60 Hz for 5 minutes. The Topi-pump was removed from the RAM unit, the stopper was carefully removed and the delivery medium in a quantity sufficient to reach a total volume of 60 ml was added to the Topi-pump. The silicone stopper was reinstalled and the sealed unit securely placed in the RAM and the Ram unit was run again at 70G for 2 minutes. The compounded medication was examined by person familiar with the art of compounding and was found to be both elegant and aesthetic. The actuator was correctly seated on the Topi-pump bottle and pouch. The contents of Topi-pump bottle were properly identified with all the information customary of compounded medications and the labeled Topi-pump was sent to an independent lab for analysis. The results from that laboratory using High Performance Liquid Chromatography (HPLC) demonstrated potency of 101% for Clonidine, 96% for Gabapentin, 102.7% for Ketoprofen, 97.7% for Lidocaine and 97.1% for Tramadol.

### Example 3

A metered dose airless pouch packaging known as a Sleekline 30 was prefilled with 16 ml of a suitable of delivery medium (in this example Versabase® was used). The closure device (a silicone stopper) was removed and 4 ml of a previously prepared and provided Progesterone aliquot (250 mg/ml) were introduced into the pouch containing the delivery medium. By calculation, this mixture will result in a topical compounded medication that has a progesterone concentration of 50 mg/ml. The closure device is restored and the stoppered Sleekline is placed securely into the Resonant acoustic mixer (RAM). The RAM unit was operated at 70G at a frequency approaching 60 Hz for 2 minutes. After which the Sleekline was removed and a 0.25 ml actuator was installed to the Sleekline bottle and pouch. The Sleekline bottle was labeled as to its contents and other information dictated by common pharmacy practices and sent to an independent laboratory to assess potency and percentage of Relative Standard Deviation (RSD). The results from the lab as it analyzed the first full actuation, the thirty-first actuation and the seventy-first actuation were the 50.5 mg/ml, 50.5 mg/ml and 51 mg/ml (respectively). This example results in a RSD of 0.47%, more than meets the USP requirement of being less or equal to 4%.

In some examples, the systems and methods disclosed herein provide improved techniques for mixing APIs in an appropriate delivery medium. In addition, the present disclosure provides systems and methods for mixing an API with a delivery agent within the same packaging as used for delivering the pharmaceutical composition for distribution. Certain embodiments disclosed herein provide for systems and methods of producing distribution-ready agents while avoiding product loss and agent exposure associated with aliquoting agents to a separate container or tube for delivery, saving time, resources and reducing exposure of a healthcare professional. Due to methods disclosed herein, a mixed product does not need to be transferred from a container to container for dispensing. Therefore, risk of exposing healthcare staff to harmful pharmaceutical agents is reduced by minimizing the time the active pharmaceutical ingredient is exposed in the mixing process and removing the transfer from one container to another. The systems and methods disclosed herein can provide greater confidence in the accuracy in dosing because the API is added and mixed directly in the packaging used for dispensing. Systems and methods disclosed herein can also be used to reduce some of the negative human factors associated with mixing or blending process by increasing uniformity or homogeneity of distribution of an API in a liquid, cream, paste or gel for example.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the above described features.

What is claimed is:

1. A method for producing a pharmaceutical composition, the method comprising:
receiving a container comprising a shell and a flexible pouch disposed within the shell, wherein the flexible pouch comprises at least one active pharmaceutical ingredient and at least one delivery agent, and wherein the flexible pouch and the shell are configured to receive a dispensing member for dispensing a metered amount of a pharmaceutical composition; and
subjecting the container to high intensity vibrations for a mixing time to produce the pharmaceutical composition.

2. The method of claim 1, wherein the flexible pouch and the shell include a single opening and wherein the method further comprises inserting a stopper into the single opening before subjecting the container to high intensity vibrations.

3. The method of claim 1, further comprising: connecting the dispensing member to the container after subjecting the container to high intensity vibrations.

4. The method of claim 1, wherein the high intensity vibrations are high intensity acoustical vibrations.

5. The method of claim 1, wherein the high intensity vibrations are from 50 gs to 100 gs.

6. The method of claim 1, wherein the mixing time is from 1 minute to 5 minutes.

7. The method of claim 1, wherein the high intensity vibrations have a frequency from 15 Hz to 1,000 Hz.

8. The method of claim 1, wherein a relative standard deviation of concentration of the at least one pharmaceutical ingredient throughout the first pharmaceutical composition is less than 4%.

9. The method of claim 1, wherein the at least one delivery agent is at least one of: glycerin, ethylene glycol, propylene glycol, mineral oil, trolamine and Emulsifix®.

10. The method of claim 1, wherein the pharmaceutical composition remains within the flexible pouch until it is provided to a user.

11. A pharmaceutical composition with a high geometric dilution, prepared by a process comprising:
placing at least one active pharmaceutical ingredient into a flexible container, wherein the flexible container is disposed within a shell and wherein the flexible container and the shell comprise an opening configured to receive a dispensing member for dispensing a metered amount of a pharmaceutical composition;
placing at least one delivery agent into the flexible container; and
subjecting the flexible container and the shell to high intensity vibrations for a mixing time, wherein the at least one active pharmaceutical ingredient and the at least one delivery agent form the pharmaceutical composition.

12. The pharmaceutical composition, according to claim 11, wherein a relative standard deviation of a concentration of the at least one active pharmaceutical ingredient throughout the pharmaceutical composition is 4% or less.

13. The pharmaceutical composition, according to claim 11, wherein the high intensity vibrations are high intensity acoustical vibrations.

14. The pharmaceutical composition, according to claim 11, wherein the high intensity vibrations are from 50 gs to 100 gs.

15. The pharmaceutical composition, according to claim 11, wherein the mixing time is from for 1 minute to 5 minutes.

16. The pharmaceutical composition, according to claim 11, wherein the high intensity vibrations have a frequency of 15 Hz to 1,000 Hz.

17. The pharmaceutical composition, according to claim 11, wherein the at least one delivery agent comprises at least one of: glycerin, ethylene glycol, propylene glycol, mineral oil, trolamine and Emulsifix®.

18. A method for producing a pharmaceutical composition for distribution, the method comprising:
receiving a container comprising a flexible pouch disposed within a container, wherein the flexible pouch comprises an opening aligned with an opening of the container and wherein the flexible pouch comprises at least one active pharmaceutical ingredient and at least one delivery agent; and
subjecting the container to high intensity vibrations for a mixing time to produce a pharmaceutical composition.

19. The method of claim 18, further comprising:
adding a second delivery agent to the flexible pouch; and
subjecting the container to second high intensity vibrations for a second mixing time to produce a second pharmaceutical composition.

20. The method of claim 18, wherein the at least one delivery agent comprises at least one of: glycerin, ethylene glycol, propylene glycol, mineral oil, trolamine and Emulsifix®

21. The method of claim 18, wherein the pharmaceutical composition is selected from the group consisting of a gel, a paste, a dense liquid or a cream.
